(19) United States
(12) Patent Application Publication
(10) Pub. No.: US 2026/0306306 A1
(43) Pub. Date: Oct. 8, 2026

(54) ELASTIC LAMINATES AND METHODS FOR ASSEMBLING ELASTIC LAMINATES FOR ABSORBENT ARTICLES

(71) Applicant: The Procter & Gamble Company, Cincinnati, OH (US)

(72) Inventors: Todd Douglas LENSER, Liberty Township, OH (US); Urmish Popatlal DALAL, Milford, OH (US); Tanner L. WILLIAMS, Loveland, OH (US)

(21) Appl. No.: 19/647,068

(22) Filed: Apr. 14, 2026

Related U.S. Application Data
(63) Continuation of application No. 19/322,850, filed on Sep. 9, 2025.
Application No. 19/322,850 is a continuation of application No. 18/485,494, filed on Oct. 12, 2023, now Pat. No. 12,440,392.
Application No. 18/485,494 is a continuation of application No. 17/368,021, filed on Jul. 6, 2021, now Pat. No. 12,070,378.
Application No. 17/368,021 is a continuation of application No. 16/049,977, filed on Jul. 31, 2018, now Pat. No. 11,083,633.
Application No. 16/049,977 is a continuation of application No. 15/674,575, filed on Aug. 11, 2017, now Pat. No. 10,561,537.
(60) Provisional application No. 62/374,010, filed on Aug. 12, 2016.
(60) Provisional application No. 62/406,025, filed on Oct. 10, 2016.
(60) Provisional application No. 62/419,515, filed on Nov. 9, 2016.

(51) Int. Cl. A61F 13/15; A61F 13/49; B29C 55/02; B29C 55/08; B29C 65/00; B29C 65/08; B29C 65/74; B29C 65/78; B29K 21/00; B29L 9/00; B29L 31/48; B32B 3/08; B32B 5/02; B32B 5/22; B32B 5/26; B32B 7/04; B32B 7/05; B32B 15/04; B32B 15/06; B32B 15/08; B32B 15/082; B32B 15/088; B32B 15/09; B32B 15/095; B32B 15/14; B32B 25/14; B32B 27/06; B32B 27/08; B32B 27/12; B32B 27/28; B32B 27/30; B32B 27/34; B32B 27/36; B32B 27/40; B32B 37/10; B32B 37/14; B32B 37/20; B32B 38/00; B32B 38/18

(52) U.S. Cl. CPC A61F 13/15699; A61F 13/15593; A61F 13/15601; A61F 13/15609; A61F 13/15674; A61F 13/15723; A61F 13/15731; A61F 13/15739; A61F 13/15764; A61F 13/49009; A61F 13/49012; A61F 13/49019; B29C 55/02; B29C 65/08; B29C 65/74; B29C 66/00145; B29C 66/43; B29C 66/4722; B29C 66/83413; B32B 5/022; B32B 7/05; B32B 27/12; B32B 37/1018; B32B 37/14; B32B 38/0012; A61F 2013/15715; A61F 2013/15869; A61F 2013/15926; A61F 2013/49093; B29C 55/08; B29C 65/086; B29C 65/7847; B29C 66/1122; B29C 66/21; B29C 66/244; B29C 66/344; B29C 66/433; B29C 66/723; B29C 66/7294; B29C 66/81469; B29C 66/83411; B29C 66/83415; B29K 2021/003; B29K 2995/0046; B29L 2009/00; B29L 2031/4878; B32B 3/08; B32B 5/22; B32B 5/26; B32B 7/04; B32B 15/04; B32B 15/043; B32B 15/06; B32B 15/08; B32B 15/082; B32B 15/088; B32B 15/09; B32B 15/095; B32B 15/14; B32B 25/14; B32B 27/06; B32B 27/08; B32B 27/285; B32B 27/302; B32B 27/34; B32B 27/36; B32B 27/40; B32B 37/144; B32B 37/20; B32B 2038/0028; B32B 38/1858; B32B 2262/0207; B32B 2262/14; B32B 2270/00; B32B 2274/00; B32B 2307/51; B32B 2307/718; B32B 2307/732; B32B 2555/02; B32B 2556/00

(57) ABSTRACT

The present disclosure relates to elastic laminates and methods for assembling elastic laminates that may be used to make absorbent article components. Elastic laminates may include one or more reinforcement layers positioned between unstretched portions of elastic materials and substrates to which the elastic materials are bonded.

### CROSS REFERENCE TO RELATED APPLICATIONS

This application is a continuation of, and claims priority under 35 U.S.C. § 120 to, U.S. patent application Ser. No. 19/322,850, filed on Sep. 9, 2025, which is a continuation of, and claims priority under 35 U.S.C. § 120 to, U.S. patent application Ser. No. 18/485,494, filed on Oct. 12, 2023, now U.S. Pat. No. 12,440,392, issued Oct. 14, 2025, which is a continuation of, and claims priority under 35 U.S.C. § 120 to, U.S. patent application Ser. No. 17/368,021, filed on Jul. 6, 2021, now U.S. Pat. No. 12,070,378, issued Aug. 27, 2024, which is a continuation of U.S. patent application Ser. No. 16/049,977, filed on Jul. 31, 2018, now U.S. Pat. No. 11,083,633, issued Aug. 10, 2021, which is a continuation of U.S. patent application Ser. No. 15/674,575, filed on Aug. 11, 2017, now U.S. Pat. No. 10,561,537, issued Feb. 18, 2020, which claims the benefit, under 35 U.S.C. § 119(e), of U.S. Provisional Patent Application Ser. No. 62/419,515, filed on Nov. 9, 2016, U.S. Provisional Patent Application Ser. No. 62/406,025, filed on Oct. 10, 2016, and U.S. Provisional Patent Application Ser. No. 62/374,010, filed on Aug. 12, 2016, the entire disclosures of which are all incorporated by reference herein.

### FIELD OF THE INVENTION

The present disclosure relates to methods for manufacturing absorbent articles, and more particularly, to elastic laminates and methods for assembling elastic laminates for making absorbent article components.

### BACKGROUND OF THE INVENTION

Along an assembly line, various types of articles, such as for example, diapers and other absorbent articles, may be assembled by adding components to and/or otherwise modifying an advancing, continuous web of material. For example, in some processes, advancing webs of material are combined with other advancing webs of material. In other examples, individual components created from advancing webs of material are combined with advancing webs of material, which in turn, are then combined with other advancing webs of material. In some cases, individual components created from advancing web or webs are combined with other individual components created from other advancing web or webs. Webs of material and component parts used to manufacture diapers may include: backsheets, topsheets, leg cuffs, waist bands, absorbent core components, front and/or back ears, and fastening components. Once the desired component parts are assembled, the advancing web(s) and component parts are subjected to a final knife cut to separate the web(s) into discrete diapers or other absorbent articles.

Some diaper components, such as leg elastics, barrier leg cuff elastics, stretch side panels, and waist elastics, are constructed from elastic laminates. Such elastic laminates may be assembled in various ways depending on the particular diaper design. For example, some elastic laminates may be constructed from one or more nonwoven substrates bonded to an elastic film. In some configurations, the elastic film may be stretched and then bonded with the nonwoven substrates to form an elastic laminate.

Some existing elastic laminate assembly operations may have certain drawbacks. For example, manufacturing operations may be configured with machines adapted to grip and stretch the films before bonding. With some gripping operations, portions of the film may remain unstretched in the assembled elastic laminate. Such unstretched portions of the film add no benefit with respect to the desired elasticity of the assembled elastic laminate. However, the unstretched portions of the film may be bonded with one or more nonwoven layers to help anchor and secure the film to the nonwoven substrates. In use, the elastic laminates may be stretched by applying forces to the elastic laminates in the regions where the unstretched portions of the film are anchored to the nonwovens. As such, when assembling elastic laminates, it may be advantageous utilize nonwovens and/or films with relatively high basis weights and/or relatively high calipers to ensure that the unstretched portions of the film and the nonwovens remain bonded together and do not separate from each other during use. However, nonwovens and/or films with relatively high basis weights can be relatively expensive and may detract from the aesthetic appearance and/or tactile impression of the assembled elastic laminate.

Consequently, it would be beneficial to provide methods and apparatuses for assembling elastic laminates that are configured with regions having a relatively high caliper where the unstretched portions of the film and nonwovens are bonded while at the same time providing the ability to construct the elastic laminate with films and/or nonwovens with relatively low basis weights.

### SUMMARY OF THE INVENTION

In one aspect, a method for assembling elastic laminates comprises the steps of: providing a first substrate and a second substrate, the first substrate and the second substrate each comprising a first surface and an opposing second surface, a first longitudinal edge and a second longitudinal edge separated from the first longitudinal edge to define a width in a cross direction; providing a first elastic material and a second elastic material, the first elastic material and the second elastic material each comprising a first edge region and a second edge region separated from the first edge region in the cross direction by a central region; stretching the central region of the first elastic material in the cross direction; stretching the central region of the second elastic material in the cross direction; advancing the first elastic material to position the stretched central region of the first elastic material in contact with the second surface of the first substrate; advancing the second elastic material to position the stretched central region of the second elastic material in contact with the second surface of the first substrate, and wherein the second edge region of the second elastic material is separated from the first edge region of the first elastic material in a cross direction; advancing the second substrate in a machine direction to position the first surface of the second substrate in contact with the stretched central regions of the first and second elastic materials; providing a first reinforcement layer between the first edge region of the first elastic material, the second edge region of the second elastic material, and either the second surface of first substrate or the first surface of the second substrate; forming an elastic laminate by ultrasonically bonding the first reinforcement layer together with the first edge region of the first elastic material, the second edge region of the second elastic material, the first substrate, and the second substrate; and cutting the elastic laminate along the machine direction through the first reinforcement layer, the first substrate, and the second substrate to form a first elastic laminate and a second elastic laminate.

In another aspect, a method for assembling elastic laminates comprises the steps of: providing a first substrate and a second substrate, the first substrate and the second substrate each comprising a first surface and an opposing second surface, a first longitudinal edge and a second longitudinal edges separated from the first longitudinal edge to define a width in a cross direction; providing a first elastic material and a second elastic material, the first elastic material and the second elastic material each comprising a first edge region and a second edge region separated from the first edge region in the cross direction by a central region; stretching the central region of the first elastic material in the cross direction; stretching the central region of the second elastic material in the cross direction; advancing the first elastic material to position the stretched central region of the first elastic material in contact with the second surface of the first substrate; advancing the second elastic material to position the stretched central region of the second elastic material in contact with the second surface of the first substrate, and wherein the second edge region of the second elastic material is separated from the first edge region of the first elastic material in a cross direction; advancing the second substrate in a machine direction to position the first surface of the second substrate in contact with the stretched central regions of the first and second elastic materials; providing a first reinforcement layer between the second edge region of the first elastic material and either the second surface of the first substrate or the first surface of the second substrate; providing a second reinforcement layer between the first edge region of the second elastic material and either the second surface of the first substrate or the first surface of the second substrate; forming an elastic laminate by ultrasonically bonding the first reinforcement layer together with the second edge region of the first elastic material, and ultrasonically bonding the second reinforcement layer together with the first edge region of the second elastic material, the first substrate, and the second substrate; and cutting the elastic laminate through the first and second substrate along the machine direction between the first elastic material and the second elastic material to form a first elastic laminate and a second elastic laminate.

In yet another aspect, a method for assembling elastic laminates comprises the steps of: providing a first substrate and a second substrate, the first substrate and the second substrate each comprising a first surface and an opposing second surface, a first longitudinal edge and a second longitudinal edges separated from the first longitudinal edge to define a width in a cross direction; wrapping the first surface of the first substrate onto an outer circumferential surface of an anvil roll; providing an elastic film, the elastic film comprising a first edge region and a second edge region separated from the first edge region in the cross direction by a central region; stretching the central region of the elastic film in the cross direction; advancing the elastic film onto the anvil roll, wherein the stretched central region of the elastic film is positioned in contact with the second surface of the first substrate; advancing a first reinforcement layer onto the anvil roll so as to be positioned between the first edge region of the elastic film and the second surface of first substrate; advancing a second reinforcement layer onto the anvil roll so as to be positioned between the second edge region of the elastic film and the second surface of first substrate; advancing the second substrate in a machine direction to position the first surface of the second substrate in contact with the stretched central region of the elastic film; ultrasonically bonding the first reinforcement layer together with the first edge region of the elastic film, the first substrate, and the second substrate; ultrasonically bonding the second reinforcement layer together with the second edge region of the elastic film, the first substrate, and the second substrate; and ultrasonically bonding the stretched central region together with the first substrate and the second substrate.

In still another aspect, an elastic laminate comprises: a first edge and a second edge extending in a longitudinal direction and separated from each other in a lateral direction; a first substrate comprising a first surface and an opposing second surface; a second substrate comprising a first surface and an opposing second surface; a film positioned between the first substrate and the second substrate, the film comprising a first edge region and a second edge region separated from the first edge region in the lateral direction by a stretchable central region, wherein the first and second end regions are laterally inboard of the first and second edges; a first reinforcement layer positioned between the first substrate and the second substrate; a second reinforcement layer positioned between the first substrate and the second substrate; a lateral stretch zone wherein the stretchable central region of the film is in direct contact with and ultrasonically bonded with the second surface of the first substrate and the first surface of the second substrate; a first reinforcement zone, wherein a first portion the first reinforcement layer extends laterally inward from the first edge to the first edge region of the film, and wherein the first portion is in direct contact with and ultrasonically bonded with the second surface of the first substrate and the first surface of the second substrate, and wherein a second portion of the first reinforcement layer extends laterally inward from the first portion to be positioned between and ultrasonically bonded with the first edge region of the film and the second surface of the first substrate; and a second reinforcement zone, wherein a first portion the second reinforcement layer extends laterally inward from the second edge to the second edge region of the film, and wherein the first portion is in direct contact with and ultrasonically bonded with the second surface of the first substrate and the first surface of the second substrate, and wherein a second portion of the second reinforcement layer extends laterally inward from the first portion to be positioned between and ultrasonically bonded with the first edge region of the film and the second surface of the first substrate.

### BRIEF DESCRIPTION OF THE DRAWINGS

FIG. 1A is a schematic side view of an apparatus for assembling an elastic laminate.

FIG. 1B is a top side view of the apparatus from FIG. 1A taken along line 1B-1B.

FIG. 1C is a left side view of the apparatus from FIG. 1B taken along line 1C-1C.

FIG. 1D is a right side view of the apparatus from FIG. 1B taken along line 1D-1D.

FIG. 1E is a detailed view of a first spreader mechanism from FIG. 1C taken along line 1E-1E.

FIG. 1F is a detailed view of a second spreader mechanism from FIG. 1D taken along line 1F-1F.

FIG. 1G is a detailed view of radially protruding nubs on an outer rim of a disk.

FIG. 1HA is a detailed view of an anvil from FIG. 1B taken along line 1HA-1HA.

FIG. 1HB is a detailed view of the anvil from FIG. 1HA taken along line 1HB-1HB.

FIG. 2A is a schematic side view of an apparatus operating to assemble elastic laminates.

FIG. 2B is a left side view of the apparatus from FIG. 2A taken along line 2B-2B.

FIG. 2C is a top side view of the first substrate advancing through a folding apparatus from FIG. 2A taken along line 2C-2C.

FIG. 2D is a detailed view of a first elastic material advancing on a first spreader mechanism from FIG. 2B taken along line 2D-2D.

FIG. 2E is a right side view of the apparatus from FIG. 2A taken along line 2E-2E.

FIG. 2F is a detailed view of a second elastic material advancing on a second spreader mechanism from FIG. 2E taken along line 2F-2F.

FIG. 3 is a top side view of an elastic laminate and apparatus from FIG. 2A taken along line 3-3.

FIG. 4 is a cross sectional view of the elastic laminate from FIG. 2A taken along line 4-4.

FIG. 4A is a cross sectional view of an alternative configuration of an elastic laminate.

FIG. 4B is a cross sectional view of a second alternative configuration of an elastic laminate.

FIG. 4C is a cross sectional view of a third alternative configuration of an elastic laminate.

FIG. 5 is an exploded cross sectional view of the elastic laminate from FIG. 4.

FIG. 5A is an exploded cross sectional view of the elastic laminate from FIG. 4A.

FIG. 5B is an exploded cross sectional view of the elastic laminate from FIG. 4B.

FIG. 5B1 is an exploded cross sectional view of the elastic laminate from FIG. 5B with a reinforcement layer formed from a Z-fold in a first substrate.

FIG. 6 is a cross sectional view of a first elastic laminate and a second elastic laminate from FIG. 2A taken along line 6-6.

FIG. 7A is a partially cut away plan view of an absorbent article in the form of a taped diaper that may include one or more elastic laminates manipulated during manufacture according to the apparatuses and methods disclosed herein with the portion of the diaper that faces away from a wearer oriented towards the viewer.

FIG. 7B is a plan view of the absorbent article of FIG. 7A that may include one or more elastic laminates manipulated during manufacture according to the apparatuses and methods disclosed herein with the portion of the diaper that faces toward a wearer oriented towards the viewer.

### DETAILED DESCRIPTION OF THE INVENTION

The following term explanations may be useful in understanding the present disclosure: “Absorbent article” is used herein to refer to consumer products whose primary function is to absorb and retain soils and wastes. “Diaper” is used herein to refer to an absorbent article generally worn by infants and incontinent persons about the lower torso. The term “disposable” is used herein to describe absorbent articles which generally are not intended to be laundered or otherwise restored or reused as an absorbent article (e.g., they are intended to be discarded after a single use and may also be configured to be recycled, composted or otherwise disposed of in an environmentally compatible manner).

An “elastic,” “elastomer” or “elastomeric” refers to materials exhibiting elastic properties, which include any material that upon application of a force to its relaxed, initial length can stretch or elongate to an elongated length more than 10% greater than its initial length and will substantially recover back to about its initial length upon release of the applied force.

As used herein, the term “joined” encompasses configurations whereby an element is directly secured to another element by affixing the element directly to the other element, and configurations whereby an element is indirectly secured to another element by affixing the element to intermediate member(s) which in turn are affixed to the other element.

The term “substrate” is used herein to describe a material which is primarily two-dimensional (i.e. in an XY plane) and whose thickness (in a Z direction) is relatively small (i.e. 1/10 or less) in comparison to its length (in an X direction) and width (in a Y direction). Non-limiting examples of substrates include a web, layer or layers or fibrous materials, nonwovens, films and foils such as polymeric films or metallic foils. These materials may be used alone or may comprise two or more layers laminated together. As such, a web is a substrate.

The term “nonwoven” refers herein to a material made from continuous (long) filaments (fibers) and/or discontinuous (short) filaments (fibers) by processes such as spunbonding, meltblowing, carding, and the like. Nonwovens do not have a woven or knitted filament pattern.

The term “machine direction” (MD) is used herein to refer to the direction of material flow through a process. In addition, relative placement and movement of material can be described as flowing in the machine direction through a process from upstream in the process to downstream in the process.

The term “cross direction” (CD) is used herein to refer to a direction that is generally perpendicular to the machine direction.

The present disclosure relates to methods for manufacturing absorbent articles, and more particularly, elastic laminates and methods for assembling elastic laminates that may be used to make absorbent article components. Particular aspects of the present disclosure involve methods for assembling an elastic laminate including a first substrate and a second substrate with a first elastic material and a second elastic material bonded between the first substrate and the second substrate. In addition, the elastic laminate may include one or more reinforcement layers positioned between unstretched portions of the elastic materials and the substrates. It is to be appreciated that in some configurations, the first and/or second elastic materials may be elastic films and/or elastic laminates, and in some configurations, the first and/or second substrates and/or reinforcement layers may be nonwovens. The first and second elastic materials are separated from each other in a cross direction and each include a first edge region and a second edge region separated from the first edge region in the cross direction by a central region, wherein the central regions are stretched in the cross direction. During assembly, one or more reinforcement layers may be positioned between the first and/or second edge regions of the first elastic material and either the first substrate or the second substrate. In addition, one or more reinforcement layers may be positioned between the first and/or second edge regions of the second elastic material and either the first substrate or the second substrate. An elastic laminate may then be formed by ultrasonically bonding the reinforcement layers together with the first edge regions the first and/or second elastic materials, the first substrate, and the second substrate. In turn, the elastic laminate may be cut along the machine direction through one or more reinforcement layers, the first substrate, and the second substrate to form a first elastic laminate and a second elastic laminate.

As discussed in more detail below, the first edge region and/or the second edge region of the first and/or second elastic materials may be unstretched when bonded with reinforcement layers and the first and/or second substrates. The reinforcement layers define areas of the elastic laminate with additional layers of material and having a relatively high caliper where the unstretched portions of the film and nonwovens are bonded. As such, the bonds located in areas where the additional material provided by the reinforcement layers are located may have relatively higher strengths to help anchor the unstretched portions of the elastic materials. In turn, the elastic laminate may be constructed with films and/or nonwovens with relatively low basis weights while the reinforcement layers may define localized regions of relatively higher calipers.

It is to be appreciated that various configurations and arrangements of apparatuses may be used to assemble elastic laminates in accordance with the methods herein. For example, the apparatuses disclosed in U.S. Patent Application No. 62/374,010, filed on Aug. 12, 2016, may be configured to assemble the elastic laminates herein. To help provide additional context to the subsequent discussion of elastic laminates and assembly configurations, the following provides a description of an apparatus that may be configured to operate in accordance with the methods disclosed herein.

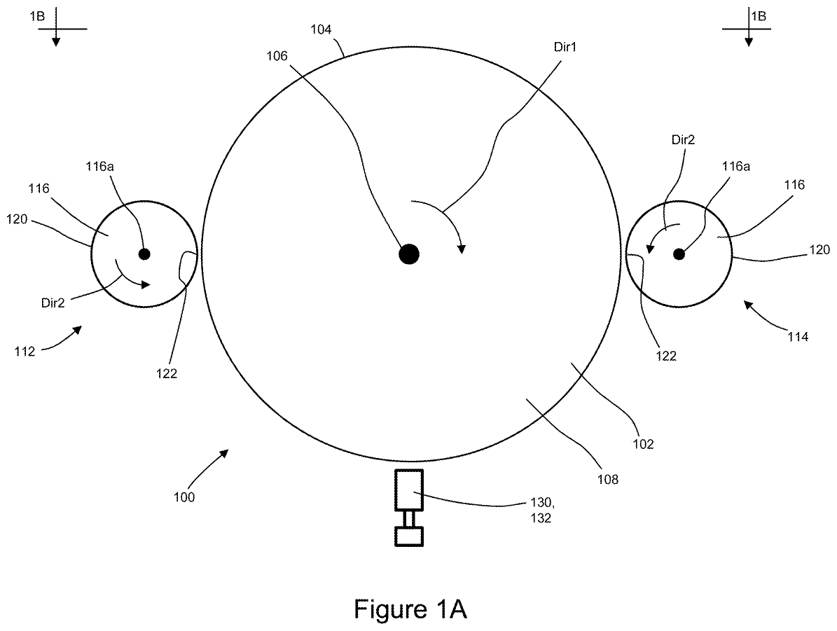

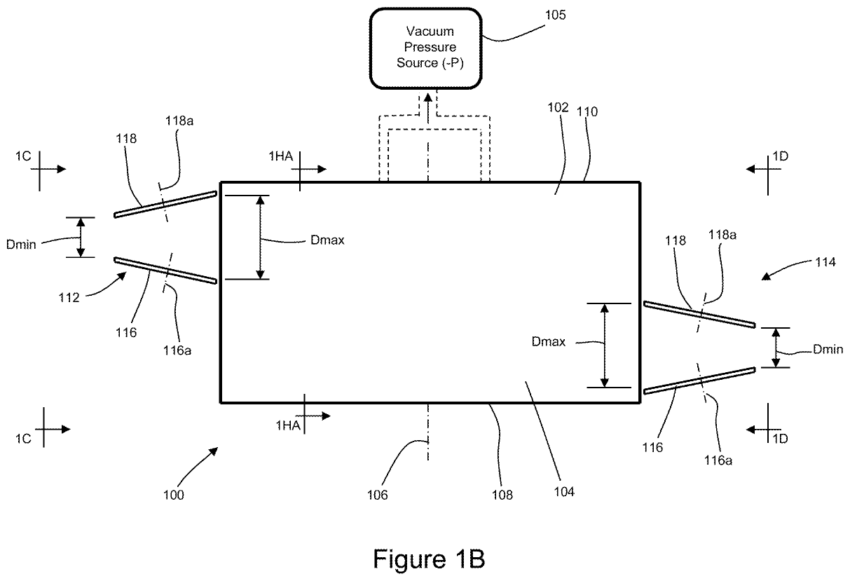

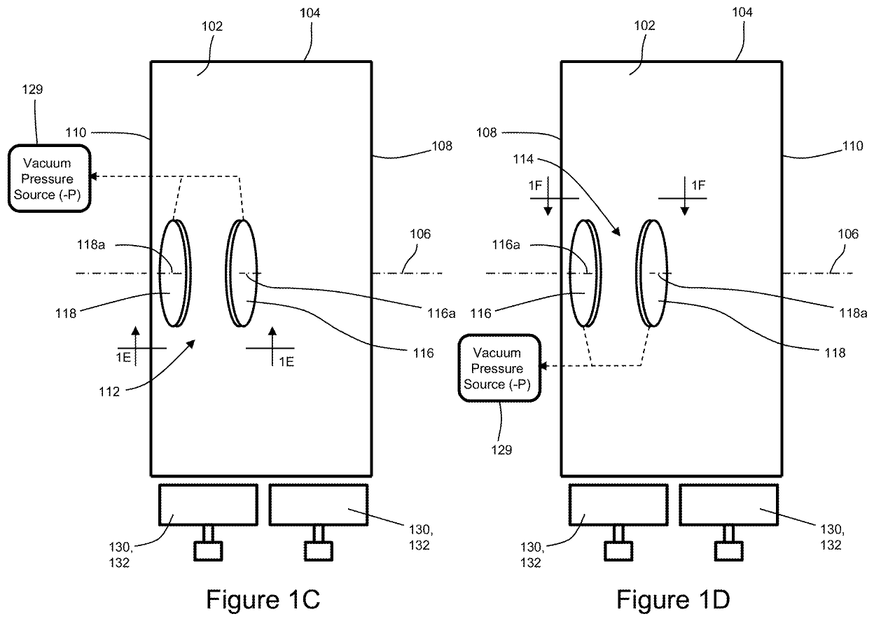

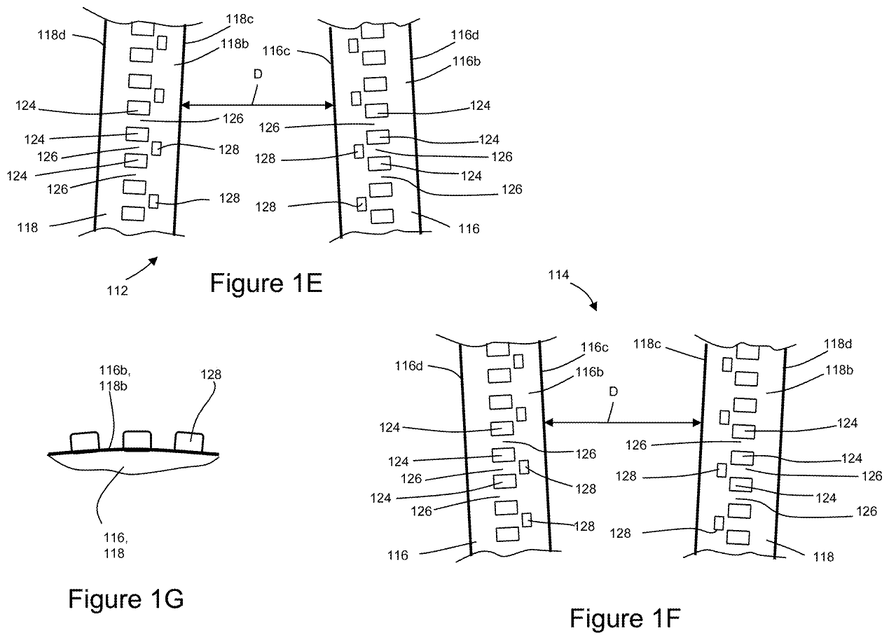

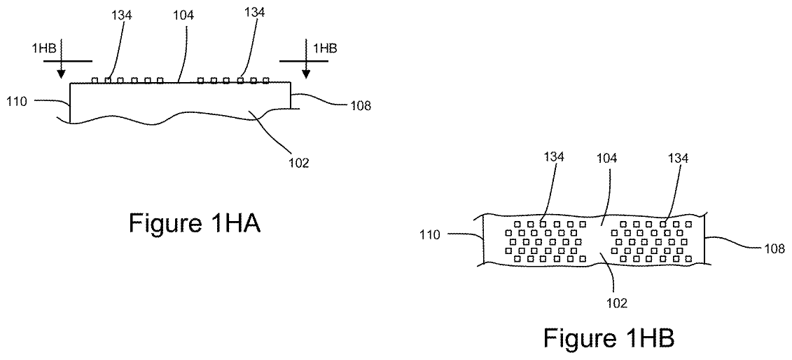

FIG. 1A-1D show schematic side views of an apparatus 100 that may be configured to assemble the elastic laminates herein. As shown in FIG. 1A-1D, the apparatus includes an anvil 102 having a cylindrically-shaped outer circumferential surface 104 and adapted to rotate in a first direction Dir1 about a first axis of rotation 106. Although the first direction Dir1 is depicted in FIG. 1A as clockwise, it is to be appreciated that the anvil 100 may be configured to rotate such that the first direction Dir1 is counterclockwise. The anvil roll 102 may extend axially for a length between a first end 108 and a second end 110. As discussed in more detail below, substrates, reinforcement layers, and elastic materials may be combined on the rotating anvil 102 to form at least one elastic laminate. It is to be appreciated that the substrates, the reinforcement layers, and the elastic materials herein may be configured in various ways. For example, the substrates and/or reinforcement materials may be configured as nonwovens, and the elastic materials may be configured as elastic films and/or elastic laminates. As shown in FIG. 1B, the anvil 102, and more particularly, the outer circumferential surface 104 may also be fluidly connected with a vacuum pressure source 105. As such, vacuum air pressure may be used to help hold the substrates, reinforcement layers, and elastic materials onto the outer circumferential surface 104 of the anvil 102 during operation.

With continued reference to FIG. 1A-1D, the apparatus 100 may also include a first spreader mechanism 112 and a second spread mechanism 114. As discussed in more detail below, the first and second spreader mechanisms 112, 114 operate to stretch elastic materials during the elastic laminate assembly process, and the stretched elastic materials are advanced from the spreader mechanisms 112, 114 onto substrates on the rotating anvil 102. As shown in FIG. 1A, the first spreader mechanism 112 may be angularly displaced from the second spreader mechanism 114 with respect to the first axis of rotation 106. As shown in FIG. 1B, the first spreader mechanism 112 may also be axially displaced from the second spreader mechanism 114 along the first axis of rotation 106.

As shown in FIG. 1A-1F, each spreader mechanism 112, 114 includes a first disk 116 and a second disk 118, wherein the first disk 116 is displaced from the second disk 118 along the axis of rotation 106. The first disk 116 is adapted to rotate about an axis of rotation 116a and the second disk 118 is adapted to rotate about an axis of rotation 118a, wherein the first and second disks 116, 118 rotate in a second direction Dir2 that is opposite the first direction Dir1. Although the second direction Dir2 is depicted in FIG. 1A as counterclockwise, it is to be appreciated that the disks 116, 118 may be configured to rotate such that the second direction Dir2 is clockwise. In addition, the first disk 116 includes an outer rim 116b extending axially between an inner edge 116c and an outer edge 116d, and the second disk 118 includes an outer rim 118b extending axially between an inner edge 118c and an outer edge 118d.

As shown in FIGS. 1A, 1B, 1E, and 1F, the first disk 116 and the second disk 118 are canted relative to each other such that the outer rims 116b, 118b are separated from each other by a distance D that increases from a minimum distance Dmin at a first location 120 to a maximum distance Dmax at a second location 122. As discussed below, elastic materials, such as elastic films, are advanced in a machine direction MD onto the outer rims 116b, 118b during operation. Because the first and second disks 116, 118 are canted, rotation of the disks 116, 118 causes the rims 116b, 118b to pull on edges regions of elastic materials and stretch the elastic materials in a cross direction CD before the elastic materials advance onto the anvil 102. As such, the disks 116, 118 may also be configured to help grip opposing edge regions of the elastic material during operation. For example, with particular reference to FIGS. 1E and 1F, the first disk 116 and the second disk 118 may each include a channel 124 extending radially inward from the rims 116b, 118b. In turn, the channels 124 may be fluidly connected with a vacuum pressure source 129. As such, vacuum air pressure may be used to help hold the elastic materials onto the rims 116b, 118b during operation. The disks 116, 118 may also include support members 126 extending across the channels 124 to the help prevent the elastic materials from being drawn into the channels 124 by the vacuum air pressure. As shown in FIGS. 1E, 1F, and 1G, the disks 116, 118 may also include nubs 128 that protrude radially outward from the rims 116b, 118b. As such, the nubs 128 may also act to help prevent the edge regions of the elastic materials from sliding along the rims 116b, 118b while stretching the elastic materials. It is to be appreciated that additional nubs 128 may be positioned inboard or outboard of the channels 124. In addition, nubs 128 may also be positioned on the support members 126.

As mentioned above, stretched elastic materials, reinforcement layers, and substrates are combined on the anvil 102. The combined substrates, reinforcement layers, and elastic materials may then be ultrasonically bonded together on the anvil 102 to form elastic laminates. As shown in FIGS. 1A, 1C, and 1D, the apparatus 100 may include one or more ultrasonic mechanisms 130 adjacent the anvil 102. It is to be appreciated that the ultrasonic mechanism 130 may include a horn 132 and may be configured to impart ultrasonic energy to the combined substrates and elastic materials on the anvil 102. As shown in FIG. 1HA and 1HB, the anvil roll 102 may include a plurality of pattern elements 134 extending radially outward from the outer circumferential surface 104 of the anvil 102. As such, the ultrasonic mechanism may apply energy to the horn 132 to create resonance of the horn at frequencies and amplitudes so the horn 132 vibrates rapidly in a direction generally perpendicular to the substrates and elastic materials being advanced past the horn 132 on the rotating anvil 102. Vibration of the horn 132 generates heat to melt and bond the substrates, reinforcement layers, and elastic materials together in areas supported by the pattern elements 134 on the anvil 102. It is to be appreciated that aspects of the ultrasonic mechanisms may be configured in various ways, such as disclosed for example in U.S. Pat. Nos. 3,113,225; 3,562,041; 3,733,238; 6,036,796; 6,508,641; and 6,645,330. In some configurations, the ultrasonic mechanism may be configured as a linear oscillating type sonotrode, such as for example, available from Herrmann Ultrasonic, Inc. In some configurations, the sonotrode may include a plurality of sonotrodes nested together in the cross direction CD.

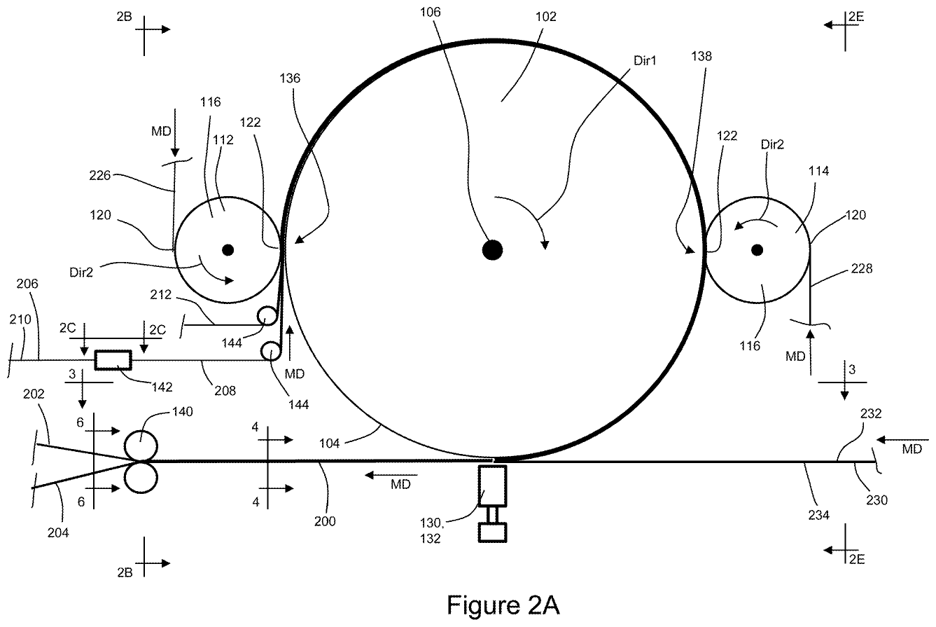

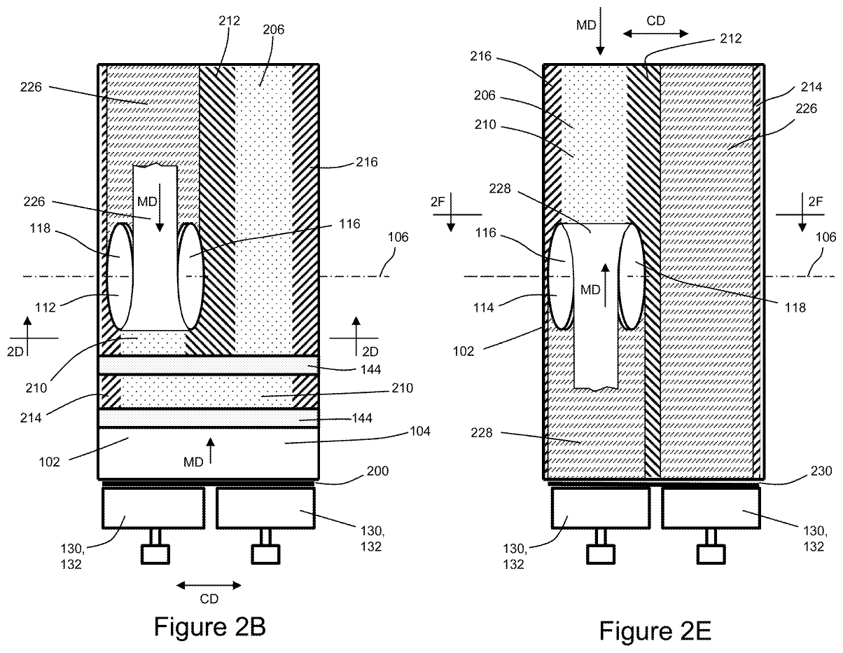

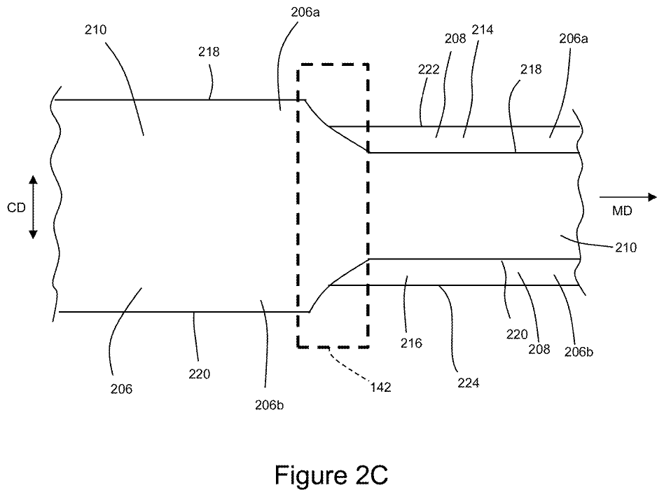

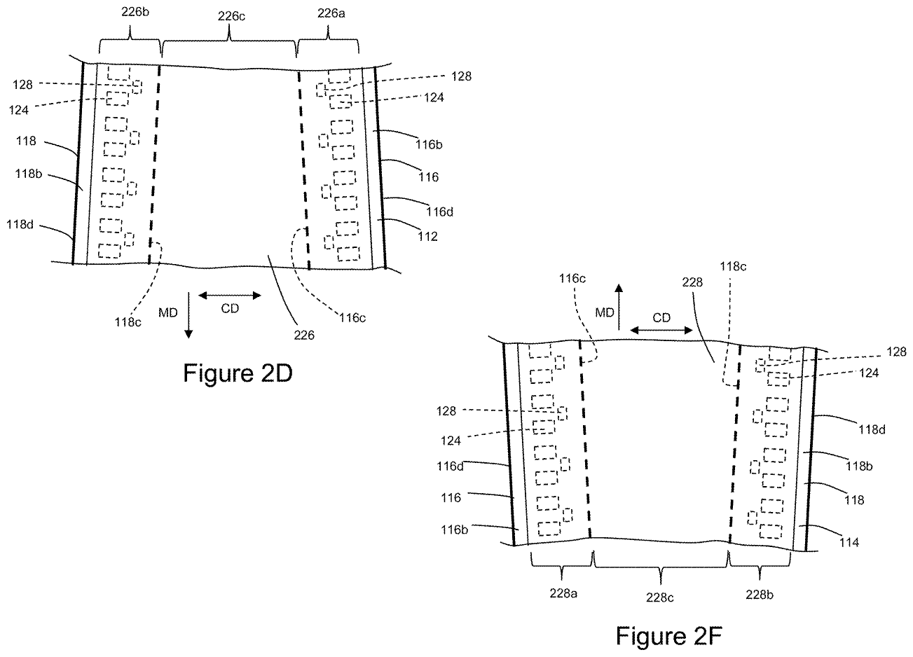

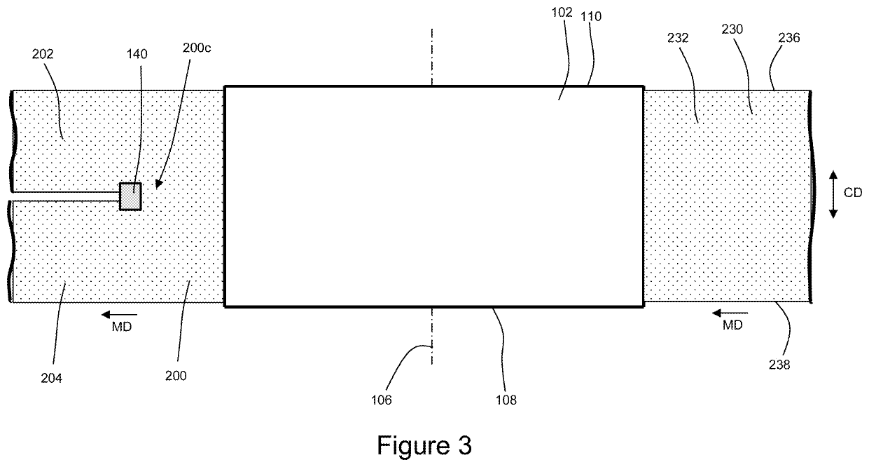

As previously mentioned, the apparatus 100 described above with reference to FIG. 1A-1HB may operate to assemble elastic laminates configured in various ways. For example, FIG. 2A-3 show various schematic views of the apparatus 100 operating to assemble an elastic laminate 200 that is subsequently slit along the machine direction MD into a first elastic laminate 202 and a second elastic laminate 204.

As shown in FIGS. 2A and 2B, a first substrate 206 advances in a machine direction MD onto the rotating anvil 102. More particularly, the first substrate 206 includes a first surface 208 and an opposing second surface 210, and the first substrate 206 advances to wrap the first surface 208 onto the outer circumferential surface 104 of the rotating anvil 102. As shown in FIGS. 2A and 2B, a first reinforcement layer 212 is advanced onto the second surface 210 of the first substrate 206. It is to be appreciated that the first reinforcement layer 212 may be formed in various ways. For example, the first reinforcement layer 212 is depicted as a discrete strip of material advanced onto the first substrate 206. With continued reference to FIG. 2B, a second reinforcement layer 214 and a third reinforcement layer 216 may also advance with the first substrate 206 onto the anvil roll 102. It is also to be appreciated that the first substrate 206 and/or the reinforcement layers 212, 214, 216 may also advance around guide rollers 144 such as shown in FIGS. 2A and 2B before advancing onto the anvil roll 102.

It is also to be appreciated that the second and third reinforcement layers 214, 216 may be formed in various ways. For example, as shown in FIG. 2C, the first substrate 206 may advance through a folding apparatus 142 that operates to fold portions of the first substrate 206 to create the second and third reinforcement layers 214, 216. In some configurations such as shown in FIG. 2C, the folding apparatus 142 may operate to fold a first longitudinal edge 218 and/or a second longitudinal edge 220 of the first substrate 206 laterally inward in the cross direction CD. More particularly, the folding apparatus 142 may fold the first substrate 206 to position a first portion 206a and a second portion 206b of the second surface 210 of the first substrate 206 in a facing relationship with the second surface 210 of the first substrate 206. As such, the folding apparatus 142 creates a first fold line 222 and a second fold line 224 in the first substrate 206 that extend in the machine direction MD. In turn, the second reinforcement layer 214 may be defined by the first portion 206a of the first substrate 206 extending between the first fold line 222 and the first longitudinal edge 218, and the third reinforcement layer 216 may be defined by the second portion 206b of the first substrate 206 extending between the second fold line 224 and the second longitudinal edge 220.

With continued reference to FIGS. 2A and 2B, during the assembly process, a first elastic material 226 is stretched in the cross direction CD and is positioned into contact with the second surface 210 of the first substrate 204, the first reinforcement layer 212, and the second reinforcement layer 214. With particular reference to FIG. 2D, the first elastic material 226 includes a first edge region 226a and a second edge region 226b separated from the first edge region 226a in the cross direction CD by a central region 226c. As shown in FIG. 2A, the first elastic material 226 advances in a machine direction MD onto the first spreader mechanism 112 at or downstream of the first location 120. In particular, the first edge region 226a of the first elastic material 226 advances onto the outer rim 116b of the first disk 116 of the first spreader mechanism 112, and the second edge region 226b advances onto the outer rim 118b of the second disk 118. As previously discussed with reference to FIG. 1E, the outer rims 116b, 118b of the first and second disks 116, 118 of the first spreader mechanism 112 may include channels 124 fluidly connected to a vacuum pressure source 129 and may include radially protruding nubs 128. Thus, as shown in FIG. 2D, the first edge region 226a of the first elastic material 226 may be held in position on the outer rim 116b with vacuum air pressure in the channels 124 and with the radially protruding nubs 128. Similarly, the second edge region 226b of the first elastic material 226 may be held in position on the outer rim 118b with vacuum air pressure in the channels 124 and with the radially protruding nubs 128.

With continued reference to FIG. 2D, as the first disk 116 and the second disk 118 of the first spreader mechanism 112 rotate, the central region 226c of the first elastic material 226 is stretched in the cross direction CD. Because the first and second edge regions 226a, 226b are held in position on the outer rims 116b, 118b, some portions of the first and second edge regions 226a, 226b may remain unstretched in the cross direction CD as the first and second disks 116, 118 rotate. Referring now to the FIGS. 2A and 2B, the first elastic material 226 advances from the first spreader mechanism 112 and is transferred onto the second surface 210 of the first substrate 206 on the anvil 102 at a first application zone 136. In particular, the stretched central region 226c of the first elastic material 226 is positioned in direct contact with the second surface 210 of the first substrate 206. In addition, the first reinforcement layer 212 is positioned between and in direct contact with the second surface 210 of the first substrate 206 and the first edge region 226a of the first elastic material 226. The second reinforcement layer 214 is also positioned between and in direct contact with the second surface 210 of the first substrate 206 and the second edge region 226b of the first elastic material 226, wherein the first longitudinal edge 218 of the first substrate 206 is positioned between the second edge region 226b of the first elastic material 226 and second surface 210 of the first substrate 206.

It is to be appreciated that during the transfer from the first spreader mechanism 112 to the anvil 102, the first elastic material 226 may be removed from the first spreader mechanism 112 at or upstream of the second location 122. As previously mentioned, the outer circumferential surface 104 of the anvil 102 may be fluidly connected with the vacuum source 105, and as such, vacuum air pressure may be applied to the first substrate 206 on the anvil 102. In addition, when the first substrate 206 is configured as a porous substrate, such as a nonwoven, vacuum air pressure may also be applied to the first elastic material 226 on the anvil 102, and as such, may help maintain the stretched condition of the central region 226c of the first elastic material 216 while on the anvil 102.

Referring now to FIGS. 2A and 2F, during the assembly process, a second elastic material 228 is stretched in the cross direction CD and is positioned into contact with the second surface 210 of the first substrate 206. With particular reference to FIG. 2F, the second elastic material 228 includes a first edge region 228a and a second edge region 228b separated from the first edge region 228a in the cross direction CD by a central region 228c. As shown in FIG. 2A, the second elastic material 228 advances in a machine direction MD onto the second spreader mechanism 114 at or downstream of the first location 120. In particular, the first edge region 228a of the second elastic material 228 advances onto the outer rim 116b of the first disk 116 of the second spreader mechanism 114, and the second edge region 228b advances onto the outer rim 118b of the second disk 118. As previously discussed with reference to FIG. 1F, the outer rims 116b, 118b of the first and second disks 116, 118 of the second spreader mechanism 114 may include channels 124 fluidly connected to a vacuum pressure source 129 and may include radially protruding nubs 128. Thus, as shown in FIG. 2F, the first edge region 228a of the second elastic material 228 may be held in position on the outer rim 116b with vacuum air pressure in the channels 124 and with the radially protruding nubs 128. Similarly, the second edge region 228b of the second elastic material 228 may be held in position on the outer rim 118b with vacuum air pressure in the channels 124 and with the radially protruding nubs 128.

With continued reference to FIG. 2F, as the first disk 116 and the second disk 118 of the second spreader mechanism 114 rotate, the central region 228c of the second elastic material 228 is stretched in the cross direction CD. Because the first and second edge regions 228a, 228b are held in position on the outer rims 116b, 118b, some portions of the first and second edge regions 228a, 228b may remain unstretched in the cross direction CD as the first and second disks 116, 118 rotate. Referring now to the FIGS. 2A and 2E, the second elastic material 228 advances from the second spreader mechanism 114 and is transferred onto the second surface 210 of the first substrate 206 on the anvil 102 at a second application zone 138. In particular, the stretched central region 228c of the second elastic material 228 is positioned in direct contact with the second surface 210 of the first substrate 206. In addition, the first reinforcement layer 212 is positioned between and in direct contact with the second surface 210 of the first substrate 206 and the second edge region 228b of the second elastic material 228. The third reinforcement layer 216 is positioned between and in direct contact with the second surface 210 of the first substrate 206 and the first edge region 228a of the second elastic material 228, wherein the second longitudinal edge 220 of the first substrate 206 is positioned between the first edge region 228a of the second elastic material 228 and second surface 210 of the first substrate 206.

As previously mentioned, the first spreader mechanism 112 is angularly displaced from the second spreader mechanism 114 with respect to the first axis of rotation 106. As such, the second application zone 138 is positioned downstream of the first application zone 136. It is to be appreciated that during the transfer from the second spreader mechanism 114 to the anvil 102, the second elastic material 218 may be removed from the second spreader mechanism 114 at or upstream of the second location 122. As previously mentioned, the outer circumferential surface 104 of the anvil 102 may be fluidly connected with the vacuum source 105, and as such, vacuum air pressure may be applied to the first substrate 206 on the anvil 102. In addition, when the first substrate 206 is configured as a porous substrate, such as a nonwoven, vacuum air pressure may also be applied to the second elastic material 228 on the anvil 102, and as such, may help maintain the stretched condition of the central region 228c of the second elastic material 228 while on the anvil 102. Also, as shown in FIG. 2E, the second elastic material 228 may be axially separated or spaced from the first elastic material 226 in the cross direction CD such that a cross directional gap exists between the first elastic material 226 and the second elastic material 228.

As shown in FIGS. 2A, 2B, and 2E, an elastic laminate 200 may be formed by ultrasonically bonding the first substrate 206, the first elastic material 226, the second elastic material 228, and the reinforcement layers 212, 214, 216 together with a second substrate 230 on the anvil 102. The second substrate 230 includes a first surface 232 and an opposing second surface 244 as well as a first longitudinal edge 236 that is separated from a second longitudinal edge 238 in the cross direction CD. The second substrate 230 advances to position the first surface 232 in contact with first elastic material 226, the second elastic material 228, the reinforcement layers 212, 214, 216, and the second surface 210 of the first substrate 206. In particular, the first edge region 226a of the first elastic material 226 and the second edge region 228b of the second elastic material 228 are positioned between the first reinforcement layer 212 and the first surface 232 of the second substrate 230. In addition, a central portion of the first reinforcement layer 212 between the first and second elastic materials 226, 228 is positioned between and in direct contact with the second surface 210 of the first substrate 206 and the first surface 232 of the second substrate 230. The first surface 232 of the second substrate 230 is also positioned in direct contact with the stretched central region 226c of the first elastic material 226 and the stretched central region 228c of the second elastic material 228. Further, the second edge region 226b of the first elastic material 226 is positioned between and in direct contact with the second reinforcement layer 214 and the first surface 232 of the second substrate 230. And the first edge region 228a of the second elastic material 228 is positioned between and in direct contact with the third reinforcement layer 216 and the first surface 232 of the second substrate 230.

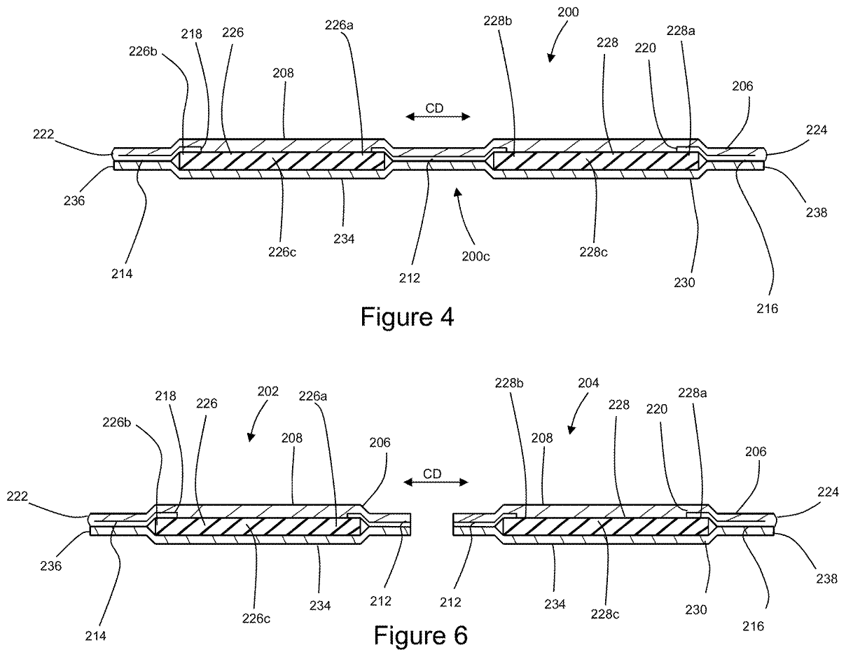

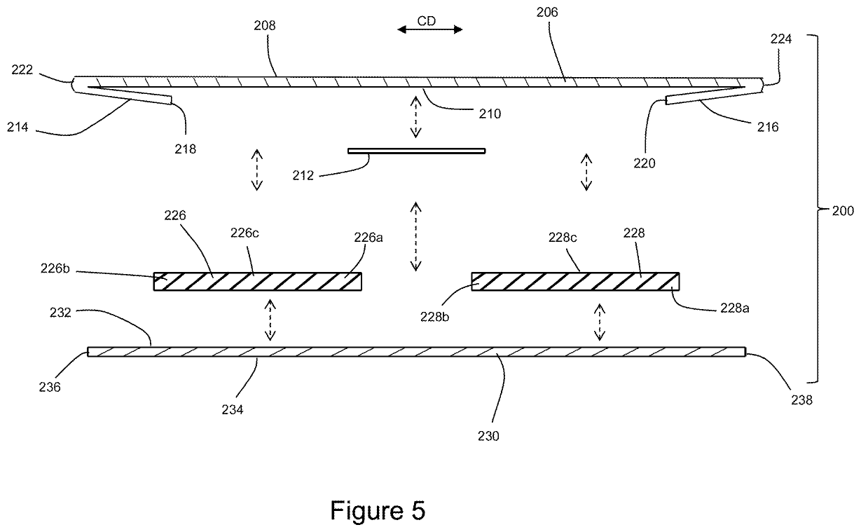

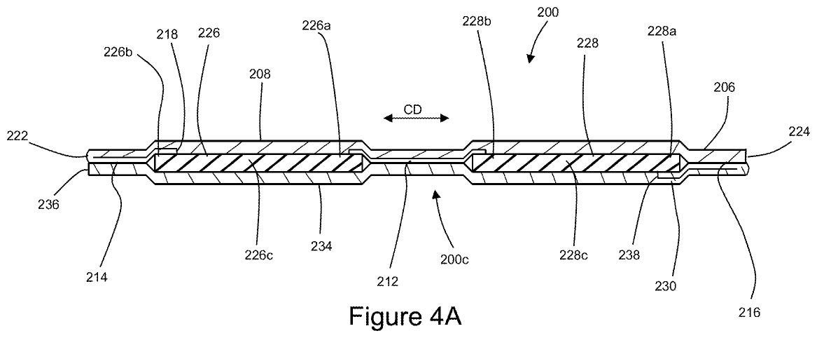

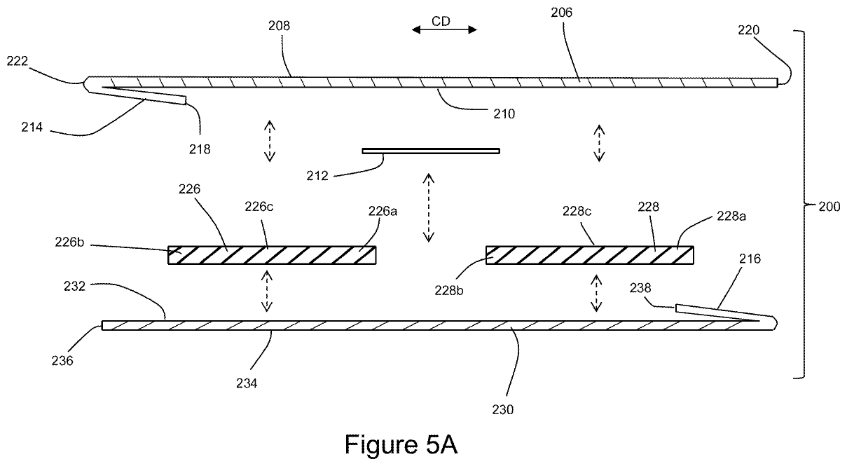

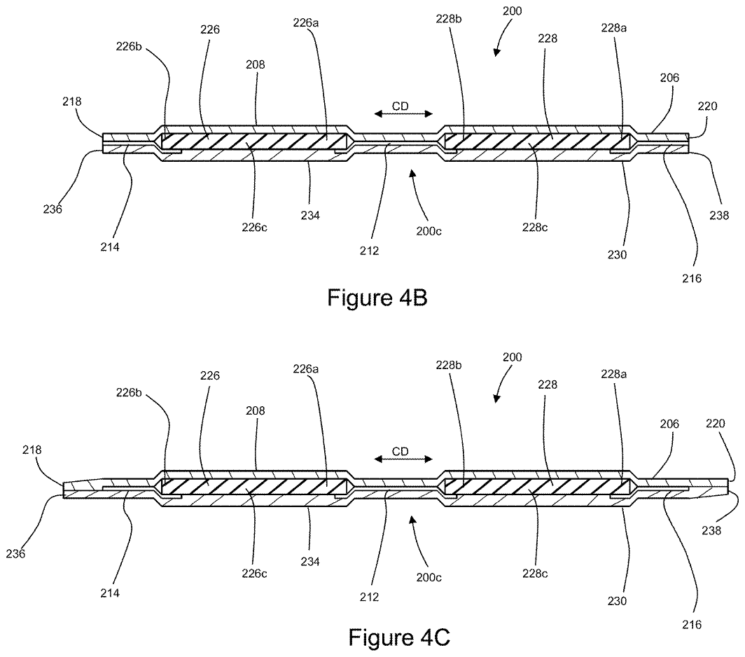

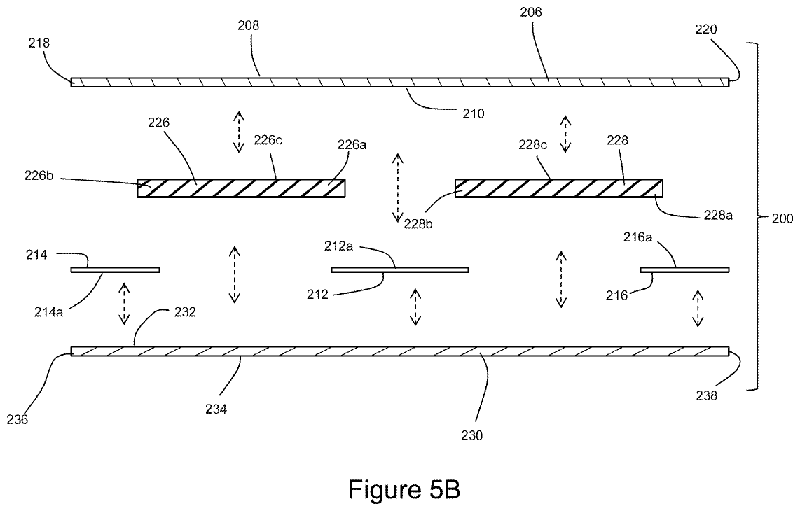

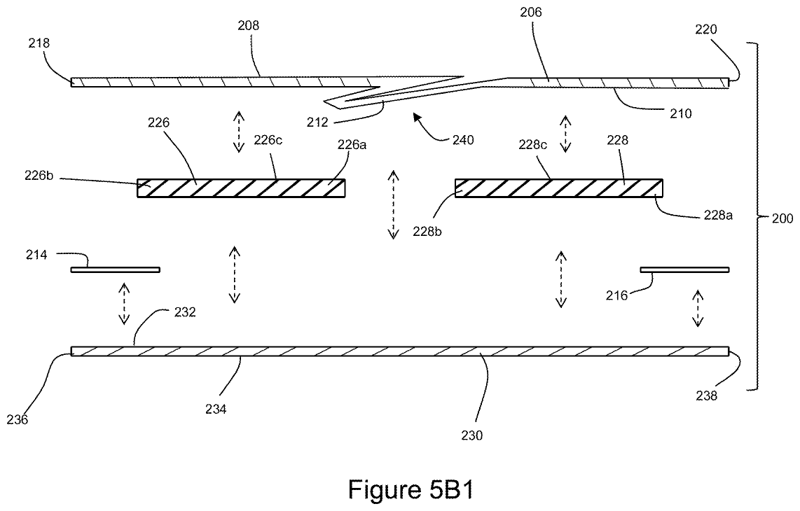

As the anvil 102 rotates, the first substrate 234, the first elastic material 216, the second elastic material 218, the second substrate 230, and the reinforcement layers 212, 214, 216 are advanced between the outer circumferential surface 104 of the anvil 102 and the ultrasonic horn 132. In turn, the ultrasonic horn 132 bonds the first substrate 206, the first elastic material 226, the second substrate 230, the first reinforcement layer 212, and the second reinforcement layer 214 together and also bonds the first substrate 206, the second elastic material 228, the second substrate 230, the first reinforcement layer 212, and the third reinforcement layer 216 together to form the elastic laminate 200, such as shown in FIGS. 4 and 5. During the ultrasonic bonding process, it is to be appreciated that bonds imparted into the elastic laminate 200 from the ultrasonic horn 132 may correspond with patterns and/or shapes defined by the plurality of pattern elements 134 extending radially outward from the outer circumferential surface 104 of the anvil 102. It is to be appreciated that the elastic laminate 200 may include various portions of components bonded together in various ways and with differing or identical bond patterns. For example, the unstretched portion of the first edge region 226a of the first elastic material 226 may be bonded together with the first substrate 206, the first reinforcement layer 212, and/or the second substrate 230. And similarly, the unstretched portion of the second edge region 228b of the second elastic material 228 may be bonded together with the first substrate 206, the first reinforcement layer 212, and/or the second substrate 230. The unstretched portion of the second edge region 226b of the first elastic material 226 may be bonded together with the first substrate 206, the second reinforcement layer 214, and/or the second substrate 230. And similarly, the unstretched portion of the first edge region 228a of the second elastic material 228 may be bonded together with the first substrate 206, the third reinforcement layer 216, and/or the second substrate 230. In addition, the stretched central region 226c of the first elastic material 226 may be bonded together with the first and/or second substrates 206, 230. Further, the stretched central region 228c of the second elastic material 228 may be bonded together with the first and/or second substrates 206, 230. Further the first substrate 206 may be bonded directly to the second substrate 230 in areas of the elastic laminate 200. It is to be appreciated that the apparatus 100 may be adapted to create various types of bond configurations, such as disclosed, for example, in U.S. Pat. No. 6,572,595.

As shown in FIGS. 2A and 6, the elastic laminate 200 may then advance from the anvil 102 to a cutter 140. In turn, the cutter 140 separates the elastic laminate 200 into the first elastic laminate 202 and the second elastic laminate 204. It is to be appreciated that the cutter 140 may be configured in various ways. For example, in some embodiments the cutter 140 may be a slitter or a die cutter that separates the elastic laminate 200 into the first elastic laminate 202 and the second elastic laminate 204. The cutter 140 may cut through the first substrate 206, the first reinforcement layer 212, and the second substrate 230 with either a straight line cut and/or a curved line cut extending in machine direction MD. The cutter 140 may also be configured as a perforator that perforates the elastic laminate 200 with a line of weakness and wherein the elastic laminate 200 is separated along the line of weakness in a later step. It is also to be appreciated that the cutter 140 may be configured to cut elastic laminate 200 into the first and second elastic laminates 202, 204 while the elastic laminate 200 is positioned on the anvil 104.

In some configurations, the cutter 140 may cut the elastic laminate 232, such as shown in FIG. 3 along a line extending in the machine direction MD through a central region or location 200c of the elastic laminate 200. As such, the elastic laminate 232 may be separated into the first elastic laminate 202 and the second elastic laminate 204, such as shown in FIG. 6. After slitting the elastic laminate 200, the first elastic laminate 202 and the second elastic laminate 204 may be allowed to relax or contract in the cross direction CD, wherein the central region 226c of the first elastic material 226 is contracted in the cross direction CD and wherein the central region 228c of the second elastic material 228 is contracted in the cross direction CD. In some configurations, the elastic laminate 200 may be allowed to relax or contract in the cross direction CD before being separated by the cutter 140 into the first elastic laminate 202 and the second elastic laminate 204.

As shown in FIGS. 3 and 4, the central region or location 200c of the elastic laminate 200 may be defined by an area between the first elastic material 226 and the second elastic material 228 where first substrate 206, the first reinforcement layer 212, and the second substrate 230 are bonded directly to each other. As such, slitting the elastic laminate 200 with the cutter 140 along the central region 200c may eliminate the need to also cut through the first elastic material 226 and/or the second elastic material 228 when creating the first and second elastic laminates 202, 204. As such, the slit edges of the first and second elastic laminates 202, 204 may not have exposed elastic material 226, 228 and thus, may be relatively more aesthetically pleasing.

It is to be appreciated that the elastic laminates 200 herein can be configured various different ways with different configurations of the first reinforcement layer 212, the second reinforcement layer 214, and the third reinforcement layer 216. For example, although the second reinforcement layer 214 and the third reinforcement layer 216 may be formed by only folding the first substrate 206 such as described above with reference to FIG. 2C, it is to be appreciated that portions of the second substrate 230 adjacent the first and second edges 236, 238 may also be folded laterally inward in the cross direction CD toward each other in addition to or alternatively to folding the first substrate 206. For example, in some configurations, the second reinforcement layer 214 may be formed by folding a portion of the first substrate 206 and/or the second substrate 230, and the third reinforcement layer 216 may be formed by folding a portion of the first substrate 206 and/or the second substrate 230. For example, as shown in FIGS. 4A and 5A, the second reinforcement layer 214 may be formed by folding a portion of the first substrate 206 along the first longitudinal edge 218, and the third reinforcement layer 216 may be formed by folding a portion of the second substrate 230 along the second longitudinal edge 238.

It is also to be appreciated that the first reinforcement layer 212, the second reinforcement layer 214, and/or the third reinforcement layer 216 may be formed by discrete strips of material in addition to or alternative to folding portions of the first substrate 206 and/or second substrate 230. For example, as shown in FIGS. 4B and 5B, the first reinforcement layer 212 may be defined by a first discrete strip of material 212a, the second reinforcement layer 214 may be defined by a second discrete strip of material 214a, and the third reinforcement layer 216 may be defined by a third discrete strip of material 216a. It is to be appreciated that the first reinforcement layer 212 and/or the second reinforcement layer 214 may be positioned between the first elastic material 226 and the first substrate 206 or the second substrate 230; and the first reinforcement layer 212 and/or the third reinforcement layer 216 may be positioned between the second elastic material 228 and the first substrate 206 or the second substrate 230. It is also to be appreciated that the first reinforcement layer 212, the second reinforcement layer 214, and/or the third reinforcement layer 216 may define varying cross directional widths and may be located in various different positions along the cross direction CD within the elastic laminate 200. For example, as shown in FIG. 4C, the second reinforcement layer 214 may not extend in the cross direction entirely to the first edges 218, 236 of the first and second substrates 206, 230, and the third reinforcement layer 216 may not extend in the cross direction entirely to the second edges 220, 238 of the first and second substrates 206, 230. In addition, the first reinforcement layer 212 may be formed by folding a portion of the first substrate 206 and/or the second substrate 230 and/or in combination with a discrete strip of material. For example, a shown in FIG. 5B1 the first reinforcement layer 212 may be formed by creating a Z-fold 240 in the first substrate 206.

It is also to be appreciated that the first reinforcement layer 212, the second reinforcement layer 214, and/or the third reinforcement layer 216 may be formed from material that is the same or different than the material of the first substrate 206 and/or second substrate 230. In some configurations, the first reinforcement layer 212, the second reinforcement layer 214, and/or the third reinforcement layer 216 may be formed from strips of material cut from the first substrate 206 and/or second substrate 230. It is also to be appreciated that the elastic laminates 200 formed herein may not include the first reinforcement layer 212, the second reinforcement layer 214, or the third reinforcement layer 216. For example, the elastic laminate 200 may include only the second and third reinforcement layers 214, 216 and may not include the first reinforcement layer 212. In another example, the elastic laminate 200 may include only the first reinforcement layer 212 and may not include the second and/or third reinforcement layers 214, 216.

It is to be appreciated that the first reinforcement layer 212, the second reinforcement layer 214, and/or the third reinforcement layer 216 may be formed from various types of materials. For example, the reinforcement layer may be a polymeric film layer that is mono-layer or multi-layer. It is to be appreciated that the polymeric material can be crystalline, semi-crystalline, or amorphous. In some configurations, the reinforcement layers may be made with polymers that are compatible with polymers of the first and/or second substrate. In some configurations, polymers may be homopolymers, co-polymers, or block co-polymers. For example, polyolefins may be used. In some configurations, polypropylene homopolymers may be compatible with polypropylene nonwoven substrates used commonly. Similarly, if the first and/or second substrate is made of polyethylene, then a reinforcement layer may be made with polyethylene. In some configurations, multi-layer film made with polypropylene core and polyethylene skins will bond strongly with polyethylene nonwovens. Polypropylene co-polymers and polyethylene co-polymers may also be suitable polymers for the reinforcement layer. Other polymers that can be used to make reinforcement layers are: styrenic polymers, thermoplastic polyurethanes, polyamids, polylactic acid, polyesters, or blends thereof.

It is to be appreciated that aspects of the methods and/or apparatus 100 herein may be configured to assemble elastic laminates from various types of material and/or components. For example, it is to be appreciated that the first substrate 206, the second substrate 230, the first reinforcement layer 212, the second reinforcement layer 214, and/or the third reinforcement layer 216 may be configured as the same or different types of materials. For example, the substrates 206, 230 and/or the reinforcement layers 212, 214, 216 may be configured as single layer or multi-layer nonwovens. In some examples wherein the elastic laminates 202, 204 may be used to manufacture diaper components, the substrate 206 may define garment facing surfaces of the elastic laminates 202, 204 in diaper components, whereas the substrate 230 may define body facing surfaces of the elastic laminates 202, 204 in diaper components. As such, the substrate 206 may be configured as a relatively high cost, premium material for aesthetic purposes, such as soft feel and appearance. In contrast, the substrate 230 may be configured as a cost optimized nonwoven, a premium nonwoven marketed as soft against a wearer's skin, or a high coefficient of friction nonwoven for improved fit. In some examples, the substrates may be configured as a relatively low basis weight nonwoven intended define a wearer facing surface, which may help to reduce the changes of pressure marks on the baby's skin from corrugations in the elastic laminates. A relatively low basis weight nonwoven may also have a relatively low bending stiffness, and thus any corrugations against the wearer's skin collapse at relatively lower forces.

As previously mentioned the first and second elastic materials 226, 228 may be configured in various ways and from various materials. For example, the elastic materials may be formed by any suitable method in the art, for example, by extruding molten thermoplastic and/or elastomeric polymers or polymer blends through a slit die and subsequently cooling the extruded sheet. Other non-limiting examples for making film forms include casting, blowing, solution casting, calendaring, and formation from aqueous or, non-aqueous cast dispersions. The elastomer composition of the present invention may be made into a film having a basis weight of from about 5 to about 150 g/m². The elastic material can also be an apertured film made of elastomeric material to provide breathability. In some configurations, the first and second elastic materials include a nonwoven web of synthetic fibers. The web can be made of fibers from elastomers or can be mixture of elastomeric fibers with plastic fibers. The first and second elastic materials may also be configured as laminates including elastic material connected with and/or interposed between an outer layer and an inner layer. The elastic material may include one or more elastic elements such as strands, ribbons, or panels. Suitable elastomeric compositions for making elastic materials comprise thermoplastic elastomers selected from the group consisting of Styrenic block copolymers, poly-esters, polyurethanes, polyether amides, polyolefin elastomers, and combinations thereof.

It is to be appreciated that aspects of the apparatus 100 herein may be configured in various ways and may operate to assemble elastic laminates 200, 202 from various types of material and/or components. For example, it is to be appreciated that the in some configurations, the elastic laminate assembly operations may be performed separate to a final assembly process, such as for example, assembling the elastic laminates offline wherein the elastic laminates may be stored until needed for production. For example, elastic laminate assembly operations may be accomplished on discrete assembly lines, separately from converting lines that may be dedicated to manufacturing disposable absorbent articles. After assemblage on the discrete lines, the elastic laminates may be delivered to the absorbent article converting lines, such as in a form of rolls of continuous elastic laminates. It is to be appreciated that such rolls of continuous elastic laminates may be planetary wound or traversely wound. It is also to be appreciated that the elastic laminate assembly process may be done online during the article assembly process.

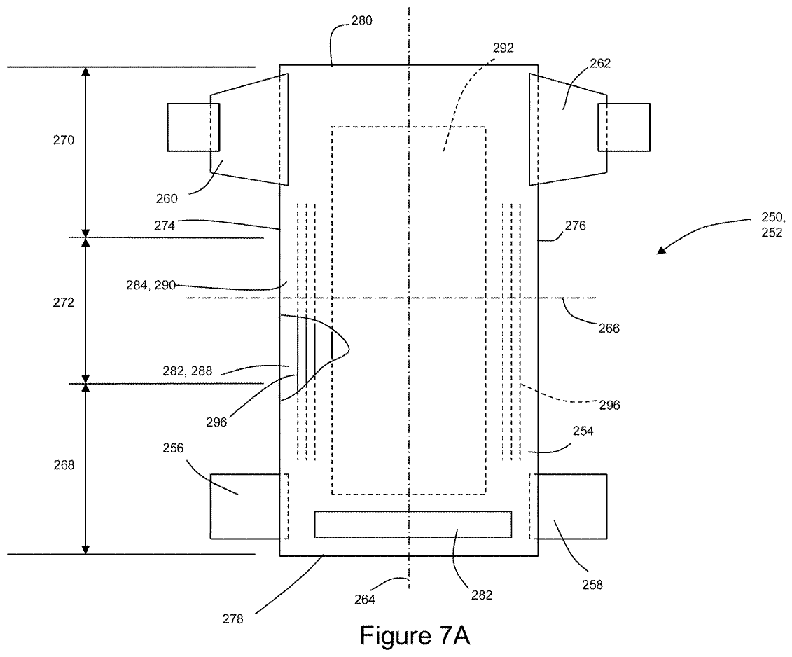

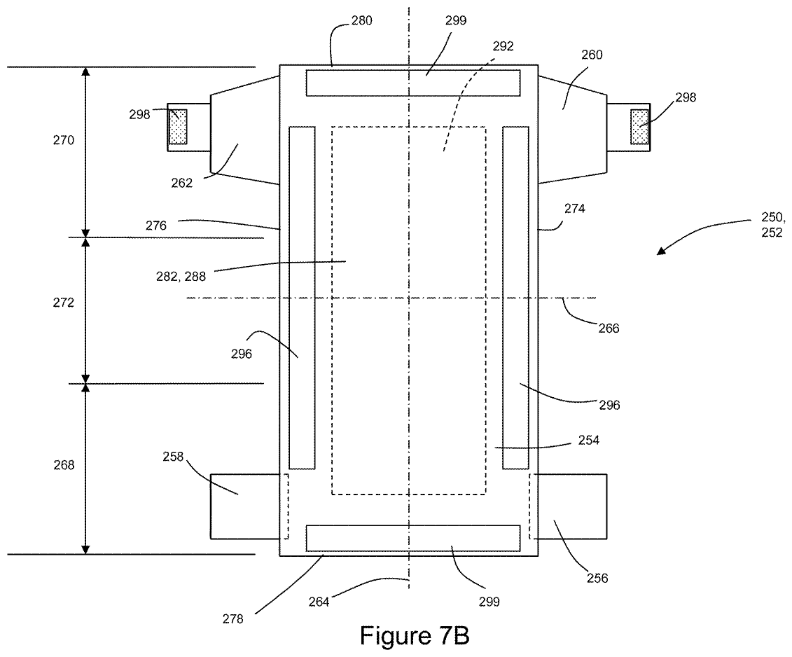

As mentioned above, apparatuses and methods of the present disclosure may be utilized to assemble various forms of elastic laminates used in the manufacture of absorbent articles. Such elastic laminates may be utilized in absorbent article components such as, for example: backsheets, topsheets, absorbent cores, front and/or back ears, fastener components, and various types of elastic webs and components such as leg elastics, barrier leg cuff elastics, and waist elastics. For the purposes of a specific illustration, FIGS. 7A and 7B show an example of a disposable absorbent article 250 in the form of a diaper 252 that may be constructed from such elastic laminates manipulated during manufacture according to the apparatuses and methods disclosed herein. In particular, FIG. 7A is a partially cut away plan view of an absorbent article in the form of a taped diaper that may include one or more elastic laminates assembled during manufacture according to the apparatuses and methods disclosed herein with the portion of the diaper that faces away from a wearer oriented towards the viewer. FIG. 7B is a plan view of the absorbent article of FIG. 7A that may include one or more elastic laminates assembled during manufacture according to the apparatuses and methods disclosed herein with the portion of the diaper that faces toward a wearer oriented towards the viewer.

As shown in FIG. 7A-7B, the diaper 252 includes a chassis 254 having a first ear 256, a second ear 258, a third ear 260, and a fourth ear 262. To provide a frame of reference for the present discussion, the chassis is shown with a longitudinal axis 264 and a lateral axis 266. The chassis 254 is shown as having a first waist region 268, a second waist region 270, and a crotch region 272 disposed intermediate the first and second waist regions. The periphery of the diaper is defined by a pair of longitudinally extending side edges 274, 276; a first outer edge 278 extending laterally adjacent the first waist region 268; and a second outer edge 280 extending laterally adjacent the second waist region 270. As shown in FIG. 7A-7B, the chassis 254 includes an inner, body-facing surface 282, and an outer, garment-facing surface 284. A portion of the chassis structure is cut-away in FIG. 7A to more clearly show the construction of and various features that may be included in the diaper. As shown in FIG. 7A-7B, the chassis 254 of the diaper 252 may include a topsheet 288 defining the inner, body-facing surface 282, and a backsheet 290 defining the outer, garment-facing surface 284. An absorbent core 292 may be disposed between a portion of the topsheet 288 and the backsheet 290. As discussed in more detail below, any one or more of the regions may be stretchable and may include an elastomeric material or laminate as described herein. As such, the diaper 252 may be configured to adapt to a specific wearer's anatomy upon application and to maintain coordination with the wearer's anatomy during wear.

The absorbent article 250 may also include an elastic waist feature 299 shown in FIG. 7B in the form of a waist band and may provide improved fit and waste containment. The elastic waist feature 299 may be configured to elastically expand and contract to dynamically fit the wearer's waist. The elastic waist feature 299 can be incorporated into the diaper and may extend at least longitudinally outwardly from the absorbent core 292 and generally form at least a portion of the first and/or second outer edges 278, 280 of the diaper 252. In addition, the elastic waist feature may extend laterally to include the ears. While the elastic waist feature 299 or any constituent elements thereof may comprise one or more separate elements affixed to the diaper, the elastic waist feature may be constructed as an extension of other elements of the diaper, such as the backsheet 290, the topsheet 288, or both the backsheet and the topsheet. In addition, the elastic waist feature 299 may be disposed on the outer, garment-facing surface 284 of the chassis 254; the inner, body-facing surface 282; or between the inner and outer facing surfaces. The elastic waist feature 299 may be constructed in a number of different configurations including those described in U.S. Patent Publication Nos. 2007/0142806A1 ; 2007/0142798A1 ; and 2007/0287983A1 , all of which are hereby incorporated by reference herein.

As shown in FIG. 7A-7B, the diaper 252 may include leg cuffs 296 that may provide improved containment of liquids and other body exudates. In particular, elastic gasketing leg cuffs can provide a sealing effect around the wearer's thighs to prevent leakage. It is to be appreciated that when the diaper is worn, the leg cuffs may be placed in contact with the wearer's thighs, and the extent of that contact and contact pressure may be determined in part by the orientation of diaper on the body of the wearer. The leg cuffs 296 may be disposed in various ways on the diaper 252.

The diaper 252 may be provided in the form of a pant-type diaper or may alternatively be provided with a re-closable fastening system, which may include fastener elements in various locations to help secure the diaper in position on the wearer. For example, fastener elements 298 may be located on the ears and may be adapted to releasably connect with one or more corresponding fastening elements located in the first or second waist regions. For example, as shown in FIG. 7A, the diaper 252 may include a connection zone 282, sometimes referred to as a landing zone, in the first waist region 268. It is to be appreciated that various types of fastening elements may be used with the diaper.

This application claims the benefit of U.S. Provisional Application Nos. 62/374,010, filed on Aug. 12, 2016; 62/406,025, filed on Oct. 10, 2016; and 62/419,515, filed on Nov. 9, 2016, the entireties of which are all incorporated by reference herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A taped absorbent article comprising:
a topsheet;
a backsheet;
an absorbent core positioned at least partially intermediate the topsheet and the backsheet;
a front waist region;
a back waist region;
a discrete ear laminate joined to the front waist region or the back waist region and extending outwardly therefrom;
wherein the discrete ear laminate comprises:
a first nonwoven substrate;
a second nonwoven substrate;
an elastic material positioned at least partially intermediate the first nonwoven substrate and the second nonwoven substrate;
a reinforcement layer positioned at least partially intermediate the first nonwoven substrate and the second nonwoven substrate, wherein the reinforcement layer comprises a section of material that is distinct from the first nonwoven substrate and the second nonwoven substrate; and
wherein the elastic material is free of overlap from itself.

2. The taped absorbent article of claim 1, wherein the reinforcement layer overlaps a portion of the elastic material.

3. The taped absorbent article of claim 1, wherein the discrete ear laminate comprises a plurality of ultrasonic bonds joining the first nonwoven substrate, the second nonwoven substrate, the elastic material, and the reinforcement layer.

4. The taped absorbent article of claim 1, wherein the reinforcement layer covers less than a total width of the discrete ear laminate in a lateral direction.

5. The taped absorbent article of claim 1, wherein the reinforcement layer terminates inboard of an outer lateral edge of the discrete ear laminate.

6. The taped absorbent article of claim 1, further comprising leg cuffs.

7. The taped absorbent article of claim 1, wherein the elastic material is generally planar and free of folds.

8. A taped absorbent article comprising:
a topsheet;
a backsheet;
an absorbent core positioned at least partially intermediate the topsheet and the backsheet;
a front waist region;
a back waist region;
a discrete ear laminate joined to the front waist region or the back waist region and extending outwardly therefrom;
wherein the discrete ear laminate comprises:
a first nonwoven substrate;
a second nonwoven substrate;
an elastic material positioned at least partially intermediate the first nonwoven substrate and the second nonwoven substrate;
a reinforcement layer positioned at least partially intermediate the first nonwoven substrate and the second nonwoven substrate, wherein the reinforcement layer comprises one or more film layers, the one or more film layers comprising two or more strips of film; and
wherein the elastic material is free of overlap from itself.

9. The taped absorbent article of claim 8, wherein the one or more film layers comprise a multilayer film having a polypropylene core and polyethylene skins.

10. The taped absorbent article of claim 8, wherein the discrete ear laminate comprises a plurality of ultrasonic bonds joining the first nonwoven substrate, the second nonwoven substrate, the elastic material, and the reinforcement layer.

11. The taped absorbent article of claim 8, wherein the reinforcement layer overlaps a portion of the elastic material.

12. The taped absorbent article of claim 8, wherein the elastic material is generally planar and free of folds.

13. The taped absorbent article of claim 8, further comprising leg cuffs.

14. A taped absorbent article comprising:
a topsheet;
a backsheet;
an absorbent core positioned at least partially intermediate the topsheet and the backsheet;
a front waist region;
a back waist region;
a discrete ear laminate joined to the front waist region or the back waist region and extending outwardly therefrom;
wherein the discrete ear laminate comprises:
a first nonwoven substrate;
a second nonwoven substrate;
an elastic material positioned at least partially intermediate the first nonwoven substrate and the second nonwoven substrate;
a reinforcement layer positioned at least partially intermediate the first nonwoven substrate and the second nonwoven substrate, wherein the reinforcement layer comprises one or more film layers and wherein the reinforcement layer covers less than a total width of the discrete ear laminate in a cross direction; and
wherein the elastic material is free of overlap from itself.

15. The taped absorbent article of claim 14, wherein the reinforcement layer comprises a separate nonwoven piece.

16. The taped absorbent article of claim 14, wherein the one or more film layers comprise two or more strips of film.

17. The taped absorbent article of claim 14, wherein the discrete ear laminate comprises a plurality of ultrasonic bonds joining the first nonwoven substrate, the second nonwoven substrate, the elastic material, and the reinforcement layer.

18. The taped absorbent article of claim 14, wherein the elastic material is generally planar and free of folds.

19. The taped absorbent article of claim 14, wherein the reinforcement layer overlaps a portion of the elastic material.

20. The taped absorbent article of claim 14, further comprising leg cuffs.
